(19) United States
(12) Patent Application Publication
(10) Pub. No.: US 2025/0387077 A1
(43) Pub. Date: Dec. 25, 2025

(54) SNORE DETECTION SYSTEM

(71) Applicant: Garmin International, Inc., Olathe, KS (US)

(72) Inventor: Arun Varadarajan, Irvine, CA (US)

(21) Appl. No.: 18/747,768

(22) Filed: Jun. 19, 2024

(51) Int. Cl. A61B 5/00; A61B 5/08; A61B 5/1455

(52) U.S. Cl. CPC A61B 5/4815; A61B 5/0816; A61B 5/14551; A61B 5/681; A61B 5/7264; A61B 5/7289; A61B 5/742; A61B 2562/0204

(57) ABSTRACT

A wearable electronic device for detecting user snoring. The wearable device can sample a microphone on a periodic basis to acquire audio data, process the audio data to identify one or more snoring candidate events and times associated with the snoring candidate events, acquire a photoplethysmography signal, process the photoplethysmography signal to identify one or more photoplethysmography events and times associated with the photoplethysmography events, and compare times associated with the snoring candidate events to the times associated with the photoplethysmography events to identify which of the snoring candidate events are actual snoring events.

### BACKGROUND

Identifying snoring can be helpful for users because it allows them to understand and manage their sleep quality and health. Typically, users must rely on complex or expensive equipment to monitor snoring, which can be cumbersome and inconvenient. Smartwatches and other wellness devices that are capable of measuring sleep metrics struggle to accurately identify snoring.

### BRIEF DESCRIPTION OF THE DRAWING FIGURES

Embodiments of the disclosure are described in detail below with reference to the attached drawing figures, wherein:

FIG. 1 depicts a view of one embodiment of an example wearable device configured for snore detection;

FIG. 2A depicts a bottom view of the wearable device of FIG. 1;

FIG. 2B depicts a system diagram showing the components of a system for carrying out embodiments of the disclosure;

FIG. 3 is a block diagram of an example process that may be utilized by embodiments of the present invention;

FIG. 4 is a first example plot showing the correlation between audio data and photoplethysmography data;

FIG. 5 is a second example plot showing the correlation between audio data and photoplethysmography data; and

FIG. 6 is a third example plot showing the correlation between audio data and photoplethysmography data.

The drawing figures do not limit the disclosure to the specific embodiments disclosed and described herein. The drawings are not necessarily to scale, emphasis instead being placed upon clearly illustrating the principles of the disclosure.

### DETAILED DESCRIPTION

The disclosure describes various embodiments of a system for detecting snoring using one or more sensors associated with a wearable device. By both detecting the presence of snoring, and that the source of the snoring is the user of the wearable device, improved sleep and snoring metrics can be provided to the user. The subject matter of embodiments of the disclosure is described in detail below to meet statutory requirements; however, the description itself is not intended to limit the scope of claims. Rather, the claimed subject matter might be embodied in other ways to include different steps or combinations of steps similar to the ones described in this document, in conjunction with other present or future technologies. Minor variations from the description below will be obvious to one skilled in the art and are intended to be captured within the scope of the claims. Terms should not be interpreted as implying any particular ordering of various steps described unless the order of individual steps is explicitly described.

The following detailed description of embodiments of the disclosure references the accompanying drawings that illustrate specific embodiments in which the disclosure can be practiced. The embodiments are intended to describe aspects of the disclosure in sufficient detail to enable those skilled in the art to practice the disclosure. Other embodiments can be utilized, and changes can be made without departing from the scope of the disclosure. The following detailed description is, therefore, not to be taken in a limiting sense. The scope of embodiments of the disclosure is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate reference to “one embodiment” “an embodiment”, or “embodiments” in this description do not necessarily refer to the same embodiment and are also not mutually exclusive unless so stated and/or except as will be readily apparent to those skilled in the art from the description. For example, a feature, structure, or act described in one embodiment may also be included in other embodiments but is not necessarily included. Thus, the technology can include a variety of combinations and/or integrations of the embodiments described herein.

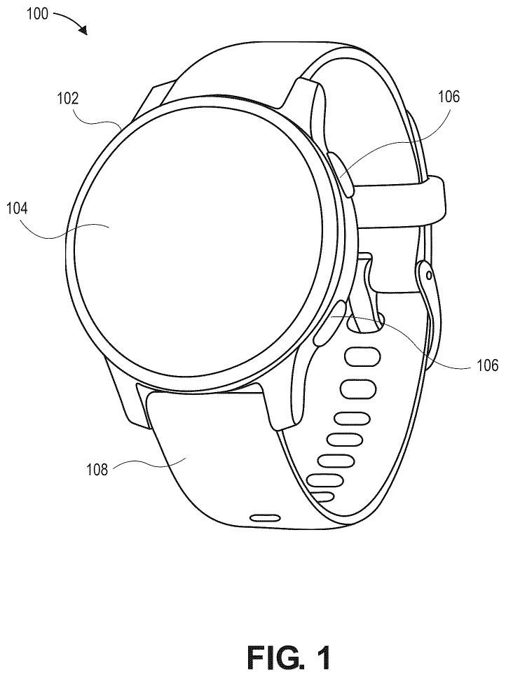

Turning first to FIG. 1, an exemplary view of one embodiment of a wearable device utilized by embodiments of the present invention is depicted. The device 100 may be configured in a variety of ways to detect and identify snoring by a wearer, The device 100 includes a housing 102 or a case configured to substantially enclose various components of the device 100. The housing 102 may be formed from a lightweight and impact-resistant material such as plastic, nylon, or combinations thereof, for example. The housing 102 may be formed from a conductive material, a non-conductive material, and combinations thereof. The housing 102 may include one or more gaskets, e.g., a seal, to make it substantially waterproof and/or water resistant. The housing 102 may include a location for a battery and/or another power source for powering one or more components of the device 100. The housing 102 may be a singular piece or may include multiple sections.

The device 100 includes a display 104 with a user interface. The display 104 may include a liquid crystal display (LCD), a thin film transistor (TFT), a light-emitting diode (LED), a light-emitting polymer (LEP), and/or a polymer light-emitting diode (PLED). The display 104 may be capable of presenting text, graphical, and/or pictorial information. The display 104 may be backlit such that it may be viewed in the dark or other low-light environments. One example embodiment of the display 104 is a 100-pixel by 64-pixel film compensated super-twisted nematic display (FSTN) including a bright white light-emitting diode (LED) backlight. The display 104 may include a transparent lens that covers and/or protects components of the device 100. The display 104 may be provided with a touch screen to receive input (e.g., data, commands, etc.) from a user. For example, a user may operate the device 100 by touching the touch screen and/or by performing gestures on the screen. In some embodiments, the touch screen may be a capacitive touch screen, a resistive touch screen, an infrared touch screen, combinations thereof, and the like. The device 100 may further include one or more input/output (I/O) devices (e.g., a keypad, buttons, a wireless input device, a thumbwheel input device, etc.). The I/O devices may include one or more audio I/O devices, such as a microphone 133, speakers, and the like. Additionally, user input may be provided from movement of the housing 102, for example, an inertial sensor(s), e.g., accelerometer, may be used to identify vertical, horizontal, angular movement and/or tapping of the housing 102 or the lens.

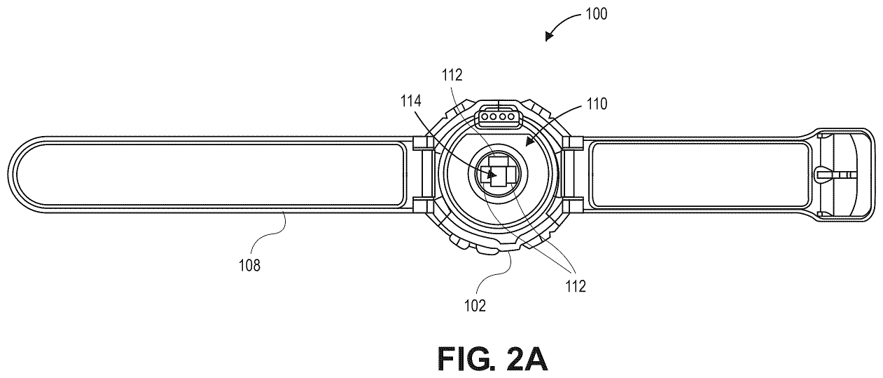

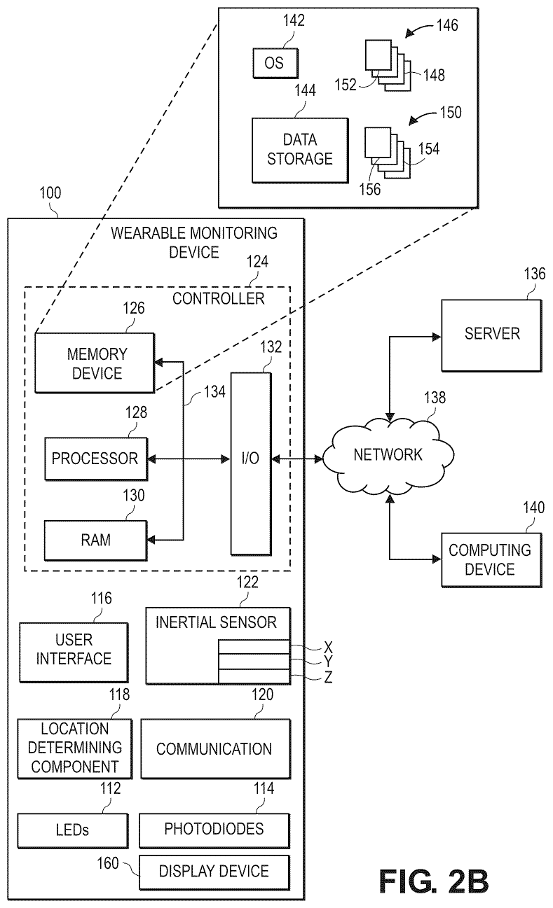

In accordance with one or more embodiments of the present disclosure, the user interface may include one or more control buttons 106. As illustrated in FIG. 1, four control buttons 106 are associated with, e.g., adjacent, the housing 102. While FIG. 1 illustrates four control buttons 106 associated with the housing 102, it is understood that the device 100 may include a greater or lesser number of control buttons 106. In one embodiment, each control button 106 is configured to generally control a function of the device 100. Functions of the device 100 may be associated with a location determining component and/or a performance monitoring component as further described below in connection with FIG. 2B. Functions of the device 100 may include, but are not limited to, displaying a current geographic location of the device 100, mapping a location on the display 104, locating a desired location and displaying the desired location on the display 104, and presenting information based on a physiological characteristic (e.g., heart-rate, heart-rate variability, blood pressure, or SpO2 percentage, PPG signal information, sleep metrics such as sleep stages, sleep quality, snoring metrics, stress level, body energy level, etc.).

FIG. 2A depicts a bottom view of one embodiment of the wearable device. The device 100 also includes a photoplethysmography (PPG) signal assembly, including one or more emitters (e.g., LEDs 112) of visible and/or non-visible light and one or more receivers (e.g., photodiodes 114) of visible and/or non-visible light that generate a light intensity signal based on the received reflection of light.

The device 100 includes a strap 108 or other attachment mechanism that enables the device 100 to be worn by a user. In particular, when the device is worn by the user, one or more LEDs and one or more photodiodes may be securely placed against the skin of a user. The strap 108 is coupled to and/or integrated with the housing 102 and may be removably secured to the housing 102 via attachment of securing elements to corresponding connecting elements. Some examples of securing elements and/or connecting elements include, but are not limited to, hooks, latches, clamps, snaps, and the like. The strap 108 may be made of a lightweight and resilient thermoplastic elastomer and/or a fabric, for example, such that the strap 108 may encircle a portion of a user without discomfort while securing the device 100 to the user. The strap 108 may be configured to attach to various portions of a user, such as a user's leg, waist, wrist, forearm, upper arm, and/or torso.

FIG. 2B depicts a system diagram showing the components of a device 100 for carrying out embodiments of the disclosure. The device 100 includes a user interface 116, a location determining component 118 (e.g., a global positioning system (GPS) receiver, assisted-GPS, etc.), a communication module 120, an inertial sensor 122 (e.g., accelerometer, gyroscope, etc.), and a controller 124. The device 100 may be a general-use wearable and mobile computing device (e.g., a watch, activity band, etc.), a cellular phone, a smartphone, a tablet computer, or a mobile personal computer, capable of monitoring a physiological characteristic and/or response of an individual as described herein. The device 100 may be a thin-client device or terminal that sends processing functions to a server 136 via a network 138. Communication via the network 138 may include any combination of wired and wireless technology. For example, the network 138 may include a USB cable between the device 100 and a computing device 140 (e.g., smartphone, tablet, laptop, etc.) to facilitate the bi-directional transfer of data between the device 100 and the computing device 140.

The controller 124 may include a memory device 126, a microprocessor (MP) 128, a random-access memory (RAM) 130, and an input/output (I/O) circuitry 132, all of which may be communicatively interconnected via an address/data bus 134. Although the I/O circuitry 132 is depicted in FIG. 2B as a single block, the I/O circuitry 132 may include a number of different types of I/O circuits. The memory device 126 may include an operating system 142, a data storage device 144, a plurality of software applications 146, and/or a plurality of software routines 150. The operating system 142 of memory device 126 may include any of a plurality of mobile platforms, such as the iOS®, Android™, Palm® webOS, Windows® Mobile/Phone, BlackBerry® OS, or Symbian® OS mobile technology platforms, developed by Apple Inc., Google Inc., Palm Inc. (now Hewlett-Packard Company), Microsoft Corporation, Research in Motion (RIM), and Nokia, respectively. The data storage device 144 of memory device 126 may include application data for the plurality of applications 146, routine data for the plurality of routines 150, and other data necessary to interact with the server 136 through the network 138. In particular, the data storage device 144 may include cardiac component data associated with one or more individuals. The cardiac component data may include one or more compilations of recorded physiological characteristics of the user, including, but not limited to, a hemoglobin saturation values, a heart rate (HR), a heart-rate variability (HRV), a blood pressure, motion data, a determined distance traveled, a speed of movement, calculated calories burned, body temperature, and the like. In some embodiments, the controller 124 may also include or otherwise be operatively coupled for communication with other data storage mechanisms (e.g., one or more hard disk drives, optical storage drives, solid state storage devices, etc.) that may reside within the device 100 and/or operatively coupled to the network 138 and/or server 136.

In some embodiments, the LEDs 112 output visible and/or non-visible light and the one or more photodiodes 114 receive transmissions or reflections of the visible and/or non-visible light and convert the received light into electrical current, which, in some embodiments, is converted into a digital value by an analog to digital converter. Each LED 112 generates light based on an intensity determined by the processor. For example, LEDs 112 may include any combination of green light-emitting diodes (LEDs), red LEDs, and/or infrared or near-infrared LEDs that may be configured by the processor to emit light into the user's skin. In some embodiments, the red LEDs operate at a wavelength between approximately 610 and 700 nm. In some embodiments, a first LED produces light at approximately 630 nm, a second LED operates at approximately 940nm, and a third LED operates at approximately 660 nm. The device 100 also includes display 104 as described in connection with FIG. 1 above.

The device 100 also includes one or more photodiodes 114 capable of receiving transmissions or reflections of visible-light and/or infrared (IR) light output by the LEDs 112 into the user's skin and generating a PPG signal based on the intensity of the reflected light received by each photodiode 114. The light intensity signals generated by the one or more photodiodes 114 may be communicated to the processor 128. In embodiments, the processor 128 includes an integrated photometric front end for signal processing and digitization. In other embodiments, the processor 128 is coupled with a photometric front end. The photometric front end may include filters for the light intensity signals and analog-to-digital converters to digitize the light intensity signals into PPG signals including a cardiac signal component associated with the user's heartbeat. Thus, the PPG signal received and utilized by the processor 128 may be filtered, modified, and transformed by various components of the device 100, including processor 128 itself, before being utilized as the PPG signal described below.

Typically, when the device 100 is worn against the user's body (e.g., wrist, fingertip, ear, etc.), the one or more LEDs 112 are positioned against the user's skin to emit light into the user's skin and the one or more photodiodes 114 are positioned near the LEDs 112 to receive light emitted by the one or more emitters after transmission through or reflection from the user's skin. The processor 128 of device 100 may receive a PPG signal based on a light intensity signal output by one or more photodiodes 114 based on an intensity of light after transmission of the light through or reflection from the user's skin that has been received by the photodiodes 114.

In both the transmitted and reflected uses, the intensity of measured light may be modulated by the cardiac cycle due to variation in tissue blood perfusion during the cardiac cycle. In activity environments, the intensity of measured light may also be strongly influenced by many other factors, including, but not limited to, static and/or variable ambient light intensity, body motion at measurement location, static and/or variable sensor pressure on the skin, motion of the sensor relative to the body at the measurement location, breathing, and/or light barriers (e.g., hair, opaque skin layers, sweat, etc.). Relative to these sources, the cardiac cycle component of the PPG signal can be very weak, for example, by one or more orders of magnitude.

The controller 124 or other elements of device 100 can calculate heart rate from the PPG signal by identifying the peaks and troughs in the electrical signal produced by photodiodes 114, which represent the systolic and diastolic phases of the cardiac cycle. By measuring the time interval between consecutive peaks, the device 100 can determine the heart rate as beats per minute. Heart rate variability (HRV), on the other hand, is calculated by analyzing the variation in time intervals between successive heartbeats detected in the PPG signal.

The PPG signal can also be used by controller 124 or other elements of device 100 to determine the respiration rate of the user by analyzing the respiratory-induced intensity variations in the blood volume, which are captured by the photodiodes 114. As the user inhales and exhales, respiratory sinus arrhythmia occurs, which is a phenomenon where the heart rate increases during inhalation and decreases during exhalation. This change in heart rate affects the blood flow dynamics, thereby influencing the light absorption and reflection detected by the photodiodes 114. By examining the periodic fluctuations in the PPG signal that correlate with the breathing cycles, the device 100 can compute the respiration rate. This computation involves detecting the frequency of these oscillations over a specified time period to determine the breaths per minute.

The location determining component 118 generally determines a current geolocation of the device 100 and may process a first electronic signal, such as radio frequency (RF) electronic signals, from a global navigation satellite system (GNSS) such as the global positioning system (GPS) primarily used in the United States, the GLONASS system primarily used in the Soviet Union, or the Galileo system primarily used in Europe. The location determining component 118 may include satellite navigation receivers, processors, controllers, other computing devices, or combinations thereof, and memory. The location determining component 118 may be in electronic communication with an antenna (not shown) that may wirelessly receive an electronic signal from one or more of the previously-mentioned satellite systems and provide the first electronic signal to location determining component 118. The location determining component 118 may process the electronic signal, which includes data and information, from which geographic information such as the current geolocation is determined. The current geolocation may include geographic coordinates, such as the latitude and longitude, of the current geographic location of the device 100. The location determining component 118 may communicate the current geolocation to the processor 128. Generally, the location determining component 118 is capable of determining continuous position, velocity, time, and direction (heading) information.

In some embodiments, the inertial sensor 122 may incorporate one or more accelerometers positioned to determine the acceleration and direction of movement of the device 100. The accelerometer may determine magnitudes of acceleration in an X-axis, a Y-axis, and a Z-axis to measure the acceleration and direction of movement of the device 100 in each respective direction (or plane). It will be appreciated by those of ordinary skill in the art that a three-dimensional vector describing a movement of the device 100 through three-dimensional space can be established by combining the outputs of the X-axis, Y-axis, and Z-axis accelerometers using known methods. Single and multiple axis models of the inertial sensor 122 may be capable of detecting magnitude and direction of acceleration as a vector quantity and may be used to sense orientation and/or coordinate acceleration of the user.

The PPG signal assembly (including LEDs 112 and photodiodes 114), location determining component 118, and the inertial sensor 122 may be referred to collectively as the “sensors” of the device 100. It is also to be appreciated that additional location determining components 118 and/or inertial sensor(s) 122 may be operatively coupled to the device 100. The device 100 may also include or be coupled to a microphone incorporated with the user interface 116 and used to receive voice inputs from the user while the device 100 monitors a physiological characteristic and/or response of the user determines physiological information based on the cardiac signal.

Communication module 120 may enable device 100 to communicate with the computing device 140 and/or the server 136 via any suitable wired or wireless communication protocol independently or using I/O circuitry 132. The wired or wireless network 138 may include a wireless telephony network (e.g., GSM, CDMA, LTE, etc.), one or more standard of the Institute of Electrical and Electronics Engineers (IEEE), such as 802.11 or 802.16 (Wi-Max) standards, Wi-Fi standards promulgated by the Wi-Fi Alliance, Bluetooth standards promulgated by the Bluetooth Special Interest Group, a near field communication standard (e.g., ISO/IEC 18092, standards provided by the NFC Forum, etc.), and so on. Wired communications are also contemplated such as through universal serial bus (USB), Ethernet, serial connections, and so forth.

The device 100 may be configured to communicate via one or more networks 138 with a cellular provider and an Internet provider to receive mobile phone service and various content, respectively. Content may represent a variety of different content, examples of which include, but are not limited to: map data, which may include route information; web pages; services; music; photographs; video; email service; instant messaging; device drivers; real-time and/or historical weather data; instruction updates; and so forth.

The user interface 116 of the device 100 may include a “soft” keyboard that is presented on the display 104 of the device 100, an external hardware keyboard communicating via a wired or a wireless connection (e.g., a Bluetooth keyboard), and/or an external mouse, or any other suitable user-input device or component. As described earlier, the user interface 116 may also include or communicate with a microphone capable of receiving voice input from a vehicle operator as well as a display device 104 having a touch input.

With reference to the controller 124, it should be understood that controller 124 may include multiple microprocessors 128, multiple RAMs 130 and multiple memory devices 126. The controller 124 may implement the RAM 130 and the memory devices 126 as semiconductor memories, magnetically readable memories, and/or optically readable memories, for example. The one or more processors 128 may be adapted and configured to execute any of the plurality of software applications 146 and/or any of the plurality of software routines 150 residing in the memory device 126, in addition to other software applications. One of the plurality of applications 146 may be a client application 152 that may be implemented as a series of machine-readable instructions for performing the various functions associated with implementing the performance monitoring system as well as receiving information at, displaying information on, and transmitting information from the device 100. The client application 152 may function to implement a system wherein the front-end components communicate and cooperate with back-end components as described above. The client application 152 may include machine-readable instructions for implementing the user interface 116 to allow a user to input commands to, and receive information from, the device 100. One of the plurality of applications 146 may be a native web browser 148, such as Apple's Safari®, Google Android™ mobile web browser, Microsoft Internet Explorer® for Mobile, Opera Mobile™ that may be implemented as a series of machine-readable instructions for receiving, interpreting, and displaying web page information from the server 136 or other back-end components while also receiving inputs from the device 100. Another application of the plurality of applications 146 may include an embedded web browser 148 that may be implemented as a series of machine-readable instructions for receiving, interpreting, and displaying web page information from the server 136 or other back-end components within the client application 152.

The client applications 146 or routines 154 may include an accelerometer routine 154 that determines the acceleration and direction of movements of the device 100, which correlate to the acceleration, direction, and movement of the user. The accelerometer routine 154 may receive and process data from the inertial sensor 122 to determine one or more vectors describing the motion of the user for use with the client application 152. In some embodiments where the inertial sensor 122 includes an accelerometer having X-axis, Y-axis, and Z-axis accelerometers, the accelerometer routine 154 may combine the data from each accelerometer to establish the vectors describing the motion of the user through three-dimensional space. In some embodiments, the accelerometer routine 154 may use data pertaining to less than three axes.

The client applications 146 or routines 150 may further include a velocity routine 156 that coordinates with the location determining component 118 to determine or obtain velocity and direction information for use with one or more of the plurality of applications, such as the client application 152, or for use with other routines.

Client applications 146 or routines 150 may also include a snore detection routine 158, that utilizes PPG signals (from photodiodes 114) and sound inputs (from microphone 133) to determine if the user of the device 100 is snoring as opposed to other nearby persons. Snore detection routine 158 is described in more detail below

The user may also launch or initiate any other suitable user interface application (e.g., the native web browser 148, or any other one of the plurality of software applications 146) to access the server 136 to implement the monitoring process. Additionally, the user may launch the client application 152 from the device 100 to access the server 136 to implement the monitoring process.

After the above-described data has been gathered or determined by the sensors of the device 100 and stored in memory device 126, the device 100 may transmit information associated with measured information (snore detection metrics, sleep metrics, etc.), peak-to-peak interval (PPI), heart rate (HR), heart-rate variability (HRV), motion data (acceleration information), location information, stress intensity level, and body energy level of the user to computing device 140 and server 136 for storage and additional processing. For example, in embodiments where the device 100 is a thin-client device, the computing device 140 or the server 136 may perform one or more processing functions remotely that may otherwise be performed by the device 100. In such embodiments, the computing device 140 or server 136 may include a number of software applications capable of receiving user information gathered by the sensors to be used in determining a physiological response (e.g., a stress level, an energy level, etc.) of the user. For example, the device 100 may gather information from its sensors as described herein, but instead of using the information locally, the device 100 may send the information to the computing device 140 or the server 136 for remote processing. The computing device 140 or the server 136 may perform the analysis of the gathered user information to determine a stress level or a body energy level of the user as described herein. The server 136 may also transmit information associated with the physiological response, such as a stress level, an energy level, of the user. For example, the information may be sent to computing device 140 or the server 136 and include a request for analysis, where the information determined by the computing device 140 or the server 136 is returned to device 100.

The disclosed techniques and described embodiments may be implemented in a wearable monitoring device having a housing implemented as a watch, a mobile phone, a hand-held portable computer, a tablet computer, a personal digital assistant, a multimedia device, a media player, a game device, arm band, or any combination thereof. The wearable monitoring device may include a processor configured for performing other activities.

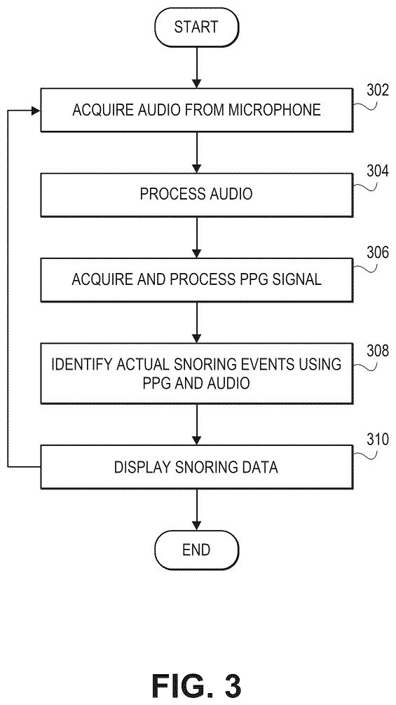

Referring now to FIG. 3, there is shown a flow chart illustrating an example process that can be performed by embodiments of device 100. In step 302, processor 128 acquires audio from the microphone. At step 304, processor 128 processes the acquired audio. At step 306, the processor 128 acquires a photoplethysmography (PPG) signal. Subsequently, at step 308, the processor 128 identifies actual snoring events for the user of device 100 using both the processed audio and the PPG signal. At step 310, the processor 128 displays the snoring data. Each of these steps will be described in greater detail below.

It should be understood that the steps illustrated in FIG. 3 and described herein can be performed in any suitable order, and are not limited to the specific sequence presented. The steps may be executed sequentially, simultaneously, or in any combination thereof. For instance, the processing of the audio (step 304) and the acquisition of the photoplethysmography (PPG) signal (step 306) may occur concurrently. Additionally, some steps may be performed iteratively or in parallel to enhance processing efficiency or to meet specific operational requirements of the device 100. Such variations and modifications in the order and combination of steps fall within the scope of embodiments of the present invention.

In step 302, the processor 128 acquires audio data from the microphone 133 by sampling the microphone on a periodic basis. The periodic sampling of audio data enables the device 100 to capture sound information relevant to detecting snoring events. The sampling process involves the processor 128 activating the microphone 133 at predefined intervals to record audio signals. These intervals, known as the sampling period, can be adjusted to optimize the device's performance and battery life. For instance, during periods of low activity or when the likelihood of snoring is minimal, the sampling period can be extended, thereby reducing the frequency of audio data acquisition and conserving battery power.

To further enhance battery conservation, the device 100 can be configured to initiate audio sampling when it determines that the user is sleeping. This determination can be made based on data from the inertial sensors 122 or the photoplethysmography (PPG) signals received from the photodiodes 114. The inertial sensors 122 can detect minimal movement, indicating that the user has likely entered a sleep state. Similarly, the PPG signals can provide information on the user's heart rate and respiratory patterns, which are indicative of sleep stages. Upon detecting that the user is asleep, the processor 128 can adjust the sampling period of the microphone 133 to a rate that balances accurate audio data acquisition with efficient power usage.

Additionally, the sampling rate of the microphone 133 can be dynamically varied based on environmental conditions and detected sound patterns. For example, the device 100 may increase the sampling rate when initial audio analysis indicates potential snoring sounds, ensuring that more detailed audio data is captured for accurate snoring event identification. Conversely, if the ambient noise level is low and no snoring is detected, the sampling rate can be decreased to conserve battery life.

The processor 128 can use default sampling rates to sample audio data from the microphone 133 that are adequate to capture snoring. For example, the processor 128 may sample the audio data every half second, every second, or every tenth of a second. The processor 128 can use default sampling rates to sample audio data from the microphone 133 that are adequate to capture snoring.

In step 304, the processor 128 processes the audio signal received from the microphone 133 to identify one or more snoring candidate events and the times associated with these events. The audio signal undergoes various signal processing techniques to extract features that are indicative of snoring. These features include amplitude, frequency, phase, and other audio components that characterize the sound patterns of snoring. By analyzing these components, the processor 128 can differentiate between potential snoring events and other types of noises. The processor 128 generates a list of snoring candidate events with associated timestamps.

The amplitude of the audio signal can be analyzed to identify the loudness of the sounds, which is a characteristic of snoring. High amplitude peaks may indicate potential snoring sounds. The frequency analysis examines the spectral content of the audio signal to identify the typical frequency ranges associated with snoring. Phase information can be analyzed to understand the periodic nature of snoring sounds. By combining these audio components, the processor 128 can identify snoring candidate events and mark the timestamps when these potential snoring sounds occur during the user's sleep.

A machine learning (ML) algorithm can be used to identify snoring candidate events in addition to as an alternative to the techniques described above. The ML algorithm is trained to recognize snoring patterns by analyzing datasets of labeled audio recordings. This algorithm can be further refined by training it on the user's own audio data, making it personalized and more accurate in identifying the user's unique snoring patterns. During training, the algorithm learns to distinguish between snoring and other noises by analyzing various features such as amplitude, frequency, and phase. Once trained, the ML algorithm can process the audio signal in real-time, or afterwards such as when the user wakes, to identify snoring candidate events. The ML algorithm can be retrained and modified through use of device 100 to adapt to any changes in the user's snoring behavior over time.

Device 100 may detect when microphone 133 is obstructed, such as when the wearable device 100 is placed under a body part, blanket, or pillow during sleep, and can adjust the acceptance thresholds for snoring detection accordingly. For instance, the thresholds required to identify snoring candidate events may be reduced when the microphone 133 is obstructed. Additionally, the machine learning techniques employed by the device may be modified to account for such obstructions. For example, the probability score used by the machine learning algorithms might be lowered to allow the identification of candidate events that would not be detected under the device's default operation. Likewise, other metrics utilized by device 100 when the microphone 133 is obstructed may include the selection of a model trained on obstructed microphone inputs or the use of lower volume or peak thresholds. Device 100 may also employ various sensors and techniques to detect obstruction of the microphone 133, such as using an ambient light sensor, skin temperature sensor, capacitance sensor, and/or the like. The ambient light sensor can detect obstructions of microphone 133 by monitoring changes in light levels, which would decrease significantly when covered by a body part, blanket, or pillow. The skin temperature sensor can be used to determine if microphone 133 is obstructed by detecting the warmth of a nearby body part, indicating that the microphone 133 is covered. The capacitance sensor can identify blockages of microphone 133 by measuring changes in electrical capacity that occur when an object, such as a hand, comes close to or covers the microphone 133.

In step 306, the processor 128 acquires the photoplethysmography (PPG) signal from the photodiode 114. The PPG signal is generated when the photodiode 114 detects changes in light intensity reflected from the user's skin, which corresponds to blood volume changes in the microvascular bed of tissue. The processor 128 then processes this raw PPG signal using various signal processing techniques and filtering techniques to remove noise and artifacts. These techniques may include low-pass filtering to eliminate high-frequency noise, band-pass filtering to isolate the relevant frequency components, and normalization to adjust the signal amplitude.

From the processed PPG signal, the processor 128 can determine the user's heart rate by identifying the peaks in the signal, which correspond to the systolic phases of the cardiac cycle. The time intervals between consecutive peaks are measured to calculate the heart rate in beats per minute (BPM). Additionally, the processor 128 can analyze the variability in these time intervals to determine the heart rate variability (HRV), which provides insights into the autonomic nervous system's regulation of the heart. HRV is calculated by examining the variations in the time intervals between successive heartbeats.

The processor 128 can also determine the user's respiration rate from the PPG signal. This is done by analyzing the respiratory-induced intensity variations in the blood volume, which are captured by the photodiode 114. As the user inhales and exhales, the heart rate exhibits respiratory sinus arrhythmia, where it increases during inhalation and decreases during exhalation. These variations influence the PPG signal. By examining the periodic fluctuations in the PPG signal that correlate with the breathing cycles, the processor 128 can compute the respiration rate by detecting the frequency of these oscillations over a specified time period, resulting in a measurement of breaths per minute.

The processor 128 can identify PPG events and the times associated with these events. PPG events include changes in heart rate, heart rate variability (HRV), respiration rate, specific times associated with inhalation and exhalation, pulse oximetry levels, stress levels, and the like. To identify these events, the processor 128 can analyze the processed PPG signal for characteristic patterns and fluctuations. For heart rate, the processor 128 detects peaks in the PPG signal, which correspond to the systolic phases of the cardiac cycle. The intervals between these peaks are measured to determine heart rate, and the timing of each peak is recorded to mark the occurrence of each heartbeat event.

The processor 128 can examine the variability in the time intervals between successive heartbeats to determine HRV. By analyzing the variations in these intervals, the processor 128 identifies HRV events and their associated times. Additionally, the processor 128 can monitor the periodic fluctuations in the PPG signal related to the user's breathing cycles. These fluctuations are used to calculate the respiration rate and identify the times associated with inhalation and exhalation. The processor 128 can mark the beginning and end of each inhalation and exhalation cycle by detecting the corresponding changes in the PPG signal amplitude and frequency, thereby associating specific times with these respiratory events.

The user's respiration rate, as determined from the PPG signal, can be used to predict the times associated with the user's next inhalation and exhalation, which are the moments when snoring is most likely to occur. The processor 128 can continuously or periodically monitor the respiration rate to identify the regular intervals of the user's breathing cycle. By predicting the timing of these inhalation and exhalation events, the processor 128 can adjust the sampling rate of the microphone 133 accordingly.

During predicted inhalation and exhalation periods, the processor 128 can increase the sampling rate of the microphone to capture more detailed audio data, enhancing the accuracy of detecting snoring events. Between these periods, the sampling rate can be reduced to conserve battery power, as the likelihood of snoring is lower.

The timing of the PPG events can be used to dynamically adjust the sampling rate of the microphone 133. By analyzing the PPG signals, the processor 128 can detect specific physiological states or changes, such as transitions in heart rate or respiration rate that may correlate with snoring. When these PPG events indicate an increased likelihood of snoring, the processor 128 can increase the sampling rate of the microphone to capture more detailed audio data, thereby improving the accuracy of snoring detection.

In step 308, the processor 128 compares the times associated with the snoring candidate events to the times associated with the photoplethysmography (PPG) events to identify which of the snoring candidate events are actual snoring events. Such functionality not only can accurately identify the existence of actual snoring events as opposed to other ambient noises, but also verify that the actual snoring events correspond to the user of the device 100 and not others nearby, such as the user's roommates or bedmates.

The processor 128 can align the timestamps of the snoring candidate events, detected through audio analysis, with the timestamps of the PPG events, such as changes in heart rate, heart rate variability (HRV), respiration rate, pulse oximetry levels, etc., to identify correlations between these datasets.

The processor 128 can analyze the temporal patterns of both the snoring candidate events and the PPG events. A significant correlation might be found if the snoring candidate events coincide with specific PPG events, such as a drop in respiration rate, pulse oximetry levels, or changes in heart rate and HRV. By synchronizing the timestamps, the processor 128 can determine if the snoring candidate events consistently occur during periods of altered PPG signals, which are indicative of physiological changes associated with snoring.

The processor 128 can employ statistical methods and machine learning algorithms to analyze the correlation between the audio and PPG data. These methods can include cross-correlation techniques to measure the similarity between the time series of snoring candidate events and PPG events. The processor 128 can use these techniques to filter out false positives, ensuring that only the snoring candidate events that show a strong correlation with the PPG events are classified as actual snoring events. This process helps in distinguishing genuine snoring from the user as opposed to other noises or sounds.

The correlation of the snoring events to the PPG events ensures that the snoring events are attributed to the user of the device 100 and not to someone else nearby, such as a roommate. By synchronizing the timestamps of the detected snoring events with the physiological data from the PPG signals, the processor 128 can verify that the identified snoring sounds coincide with the user's physiological responses, such as changes in heart rate, heart rate variability, respiration rate, pulse oximetry levels, etc. This correlation confirms that the snoring events are directly associated with the user's biometric data, thereby distinguishing the user's snoring from any external sounds or snoring from other individuals in proximity. Likewise correlating PPG events with snoring events reduces false positives, such as loud noises that might be inadvertently identified as snoring events through audio processing alone.

Changes in a user's pulse oximetry (SpO2) and respiration rate can be correlated with candidate snoring events to identify actual snoring events. The processor 128 can monitor the SpO2 levels and respiration rate using the PPG signals from the photodiodes 114. A drop in SpO2 levels, often indicative of restricted airflow, combined with irregularities or patterns in the respiration rate, can signal the presence of snoring. By aligning these physiological changes with the timestamps of candidate snoring events detected through audio analysis, the processor 128 can more accurately determine which candidate events are actual snoring events.

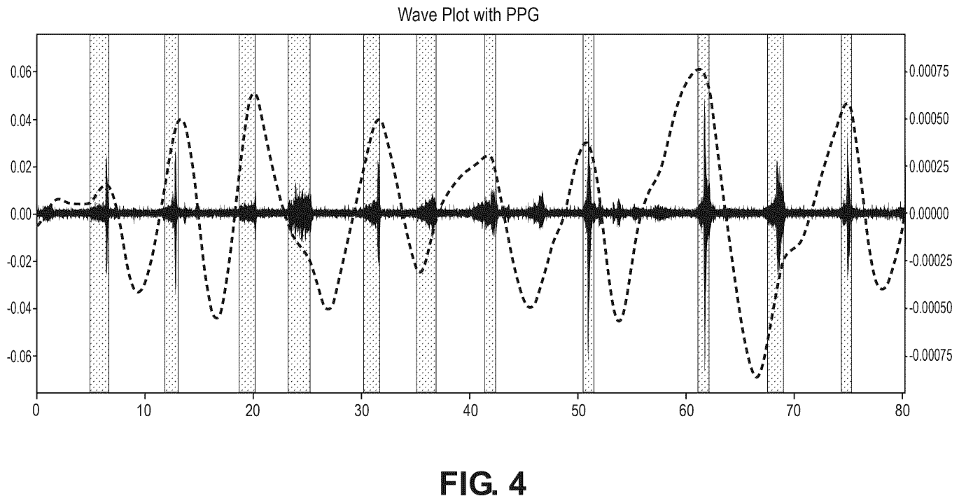

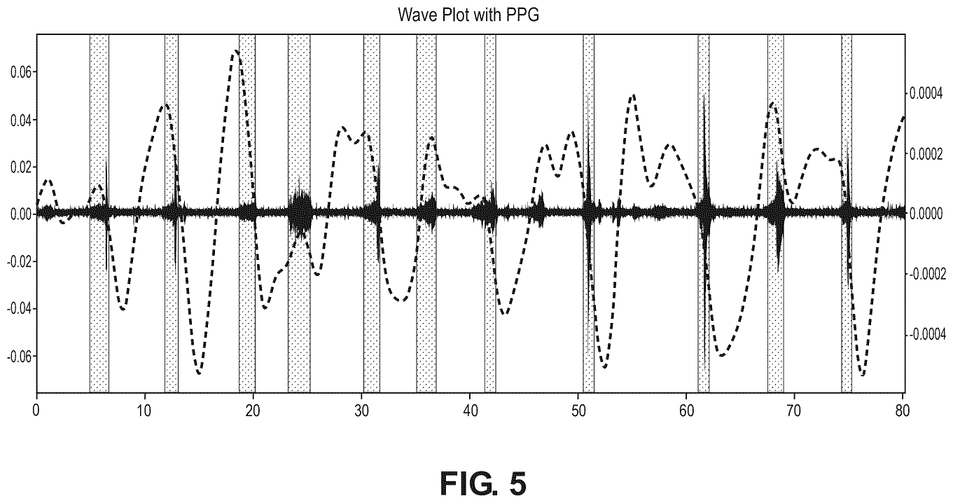

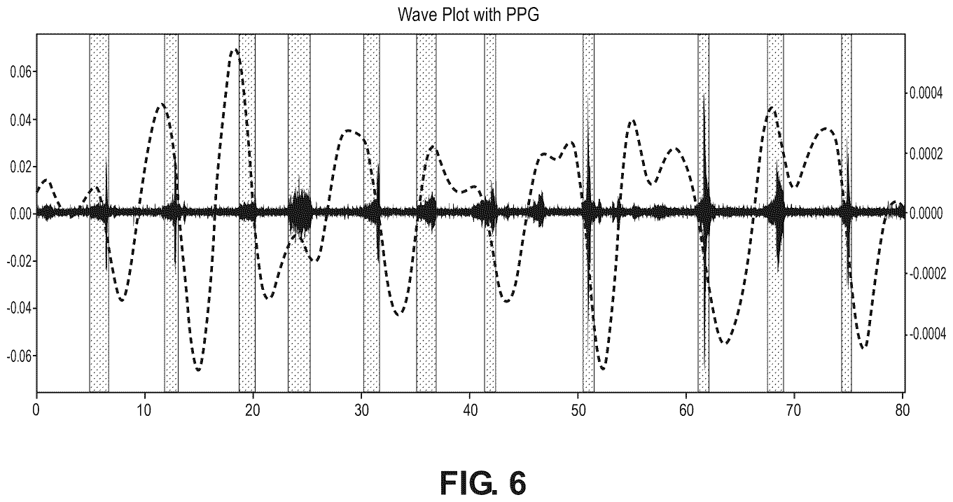

Referring now to FIGS. 4-6, example PPG signals and corresponding audio data plots are illustrated. FIG. 4 shows the correlation between a PPG signal and audio recordings of a person snoring. FIG. 5 shows a derivative of an example PPG signal and its correlation to an audio signal of person snoring. FIG. 6 shows a derivative of an example PPG signal, with a bandpass filter applied to remove heart pulse waves, and its correlation to an audio signal of a person snoring. FIGS. 4-6 are merely examples of the type of correlations between PPG signals and audible snoring.

Inputs from the inertial sensors 122 can be used to supplement the candidate snoring events and PPG events to identify actual snoring events. The inertial sensors, which detect acceleration and vibrations, can capture the physical movements and vibrations associated with snoring. When the processor 128 detects vibrations that coincide with the audio data from the microphone 133 and the physiological data from the PPG signals, it can increase the confidence in identifying a snoring event. The acceleration data provides an additional layer of verification, ensuring that the detected snoring is not a false positive caused by external noise or other sources. In some embodiments, inputs from the inertial sensors 122 may be used instead of audio signals from the microphone 133 or PPG signals from the photodiodes 114.

In step 310, the processor 128 uses the identified actual snoring events to generate detailed snoring data. This data may include the times of occurrence, duration, and frequency of snoring events throughout the night. The processor 128 can analyze these snoring events to provide insights into the user's snoring patterns. The generated snoring data can include metrics such as the number of snoring events per hour, the average decibel level of the snoring, and any correlations with physiological changes detected by the PPG signals, such as heart rate variability and respiration rate.

The snoring data can be compiled into a snoring report, which can be displayed on the display 104 of device 100. The report can present detailed information about the user's snoring patterns, including the times of occurrence and duration of each snoring event. It also can provide insights into how snoring affects the user's physiological state, such as changes in heart rate and respiration rate during snoring episodes. This report can be accessed by the user in the morning or at any convenient time to review their sleep quality and snoring metrics.

The device 100 can present snoring data on the display 104 in real-time or after the user wakes up. In real-time, the device 100 can show ongoing snoring events, allowing the user to be aware of their snoring patterns as they occur. Upon waking, the user can view a summary of the night's snoring data, providing an overview of their snoring behavior and any associated physiological changes. This real-time and post-sleep presentation of data helps the user monitor their snoring and understand its impact on their overall sleep quality.

The processor 128 can calculate the severity of identified actual snoring events over the course of a night by analyzing various factors. This calculation may include the number of actual snoring events, the duration of each event, and the total duration of all snoring events. Additionally, the processor 128 can evaluate the impact of snoring on physiological metrics, such as changes in pulse oximetry (SpO2) levels, heart rate variability (HRV), and other sleep metrics. By combining these factors, the processor 128 may generate a severity score that reflects the overall impact of snoring on the user's sleep quality and health. This severity score can be provided to the user via display 104 upon waking, as part of a sleep report, or otherwise stored for later access and analysis by the user.

Additionally, the processor 128 can generate alerts to wake the user upon detecting snoring events or when certain thresholds associated with snoring events are met. For example, if the number of snoring events exceeds a predefined threshold per hour, or if the average decibel level of snoring is high, the device 100 can vibrate or emit a sound to alert the user. Alerts can also be triggered by changes in PPG events, such as decreasing pulse oximetry levels, indicating potential health risks. These alerts help the user address snoring issues promptly, potentially improving sleep quality and reducing health risks associated with prolonged snoring.

In addition to generating and displaying snoring data, the device 100 can share this information using the communication module 120. The communication module 120 enables the device to transmit snoring data to a server 136, where it can be correlated with other user data or health data. By aggregating and analyzing data from multiple sources, the server 136 can provide a more integrated view of the user's health. For instance, snoring data can be correlated with data from other wearable devices, medical records, or lifestyle information to identify patterns or potential health concerns.

The server 136 can then share this correlated health data with the user through various interfaces such as smartphones, web pages, or other devices. Users can access their snoring and health data through dedicated mobile apps or web portals, providing them with a convenient way to monitor and manage their health. Healthcare providers can also access this information, enabling them to offer personalized advice or interventions based on the user's snoring patterns and overall health profile.

The server 136, device 100, or other services can utilize the snoring data generated by the processor 128 to compare and correlate it with other data generated by the device 100, including activity data and wellness data. By analyzing these correlations, the system can identify patterns and associations between snoring events and the user's overall health and behavior. For example, the server 136 can compare the frequency and intensity of snoring events with the user's activity levels, sleep patterns, and other physiological metrics recorded throughout the day and night.

One possible correlation might be between low heart rate variability (HRV) before sleep and an increased number of snoring events. By analyzing HRV data captured by the PPG signals from the photodiode 114, the processor 128 can determine periods of high stress or low relaxation before the user goes to bed. The server 136 can then compare this data with the snoring events recorded during the night to identify any patterns. For instance, it might be observed that nights with low HRV before sleep correspond to a higher frequency of snoring events, suggesting a potential link between pre-sleep stress levels and snoring.

Additionally, the server 136 can analyze the relationship between the user's daily physical activity and snoring events. Activity data captured by the inertial sensors 122, such as the duration and intensity of physical exercise, can be correlated with the snoring data. The analysis can reveal that intense physical activity during the day is associated with a reduction in the number of snoring events at night. By comparing periods of high physical activity with the corresponding snoring data, the server 136 can identify trends that suggest to the user how daytime behaviors influence nighttime snoring patterns.

A smartphone paired with device 100 via the communication module 120 can be used to record audio data to save the battery life of device 100. When device 100 identifies PPG events that suggest potential snoring, it can signal the paired smartphone to begin recording audio data. Additionally or alternatively, the smartphone can begin sampling audio data once the device 100 indicates that the user is asleep based on the PPG events and/or data from the inertial sensors 122. The smartphone, with its typically larger battery capacity, can handle the audio recording, thereby conserving the battery of device 100. The recorded audio data, along with the PPG events detected by device 100, can later be analyzed by device 100, the smartphone, and/or server 136 to identify actual snoring events. This combined data can be used to generate detailed snoring data, correlating the physiological changes detected by device 100 with the audio recordings from the smartphone to generate snoring data.

Having thus described various embodiments, what is claimed as new and desired to be protected by Letters Patent includes the following:

1. A wearable electronic device configured to be worn by a user, the device comprising:
a display;
a light emitting diode configured to emit light into the user's body;
a photodiode configured to detect light reflected from the user's body and to generate a photoplethysmography signal;
a microphone; and
a processor coupled with the display, the light emitting diode, the photodiode, and the microphone, the processor configured to:
sample the microphone on a periodic basis to acquire audio data;
process the audio data to identify one or more snoring candidate events and times associated with the snoring candidate events;
acquire the photoplethysmography signal from the photodiode;
process the photoplethysmography signal to identify one or more photoplethysmography events and times associated with the photoplethysmography events;
compare times associated with the snoring candidate events to the times associated with the photoplethysmography events to identify which of the snoring candidate events are actual snoring events corresponding to the user; and
control the display to present data corresponding to the actual snoring events.

2. The device of claim 1, wherein the one or more photoplethysmography events include user respiration.

3. The device of claim 2, wherein the one or more photoplethysmography events include inhalation and exhalation times of the user.

4. The device of claim 1, wherein the one or more photoplethysmography events include a pulse oximetry level.

5. The device of claim 4, wherein the one or more photoplethysmography events include a drop in pulse oximetry level.

6. The device of claim 1, wherein the processor is further operable to control the sampling rate of the microphone.

7. The device of claim 6, wherein the processor is operable to control the sampling rate of the microphone based on the one or more identified photoplethysmography events.

8. The device of claim 1, wherein the processor is further operable to alert the user upon identification of one or more actual snoring events.

9. The device of claim 1, wherein the processor is configured to control the display to indicate a severity of the actual snoring events.

10. A wearable electronic device configured to be worn by a user, the device comprising:
a display;
a light emitting diode configured to emit light into the user's body;
a photodiode configured to detect light reflected from the user's body and to generate a photoplethysmography signal;
a microphone; and
a processor coupled with the display, the light emitting diode, the photodiode, and the microphone, the processor configured to:
sample the microphone on a periodic basis to acquire audio data;
process the audio data to identify one or more snoring candidate events and times associated with the snoring candidate events;
acquire the photoplethysmography signal from the photodiode;
process the photoplethysmography signal to identify one or more photoplethysmography events and times associated with the photoplethysmography events, the one or more photoplethysmography events including user respiration;
compare times associated with the snoring candidate events to the times associated with the user respiration to identify which of the snoring candidate events are actual snoring events corresponding to the user;
control the sampling rate of the microphone based on user respiration; and
control the display to present data corresponding to the actual snoring events.

11. The device of claim 10, wherein the one or more photoplethysmography events include inhalation and exhalation times of the user.

12. The device of claim 10, wherein the one or more photoplethysmography events include a pulse oximetry level.

13. The device of claim 10, wherein the one or more photoplethysmography events include a drop in pulse oximetry level.

14. The device of claim 10, wherein the processor is further operable to alert the user upon identification of one or more actual snoring events.

15. The device of claim 10, wherein the processor is configured to control the display to indicate a severity of the actual snoring events.
